(19) United States
(12) Patent Application Publication
(10) Pub. No.: US 2024/0282467 A1
(43) Pub. Date: Aug. 22, 2024

(54) METHOD AND DEVICE FOR ANALYZING INTERACTIONS BETWEEN DRUGS

(71) Applicant: DR.NOAH BIOTECH INC., Suwon-si, Gyeonggi-do (KR)

(72) Inventors: Ji Hyun LEE, Suwon-si, Gyeonggi-do (KR); Sang Hun BAE, Incheon (KR); Jung Kyu CHOI, Seoul (KR); In Seop LEE, Suwon-si, Gyeonggi-do (KR)

(21) Appl. No.: 18/567,683

(22) PCT Filed: Mar. 17, 2022
(86) PCT No.: PCT/KR2022/095058

§ 371 Date: Dec. 6, 2023

(30) Foreign Application Priority Data
Jun. 16, 2021 (KR) 10-2021-0078199

(51) Int. Cl. G16H 70/40; G06N 20/00

(52) U.S. Cl. CPC G16H 70/40; G06N 20/00

(57) ABSTRACT

A method for analyzing drug-drug interaction includes: acquiring a first data set for chemical structures of drugs, a second data set for a grade of a side effect between the drugs and a third data set for a type of a side effect between the drugs, generating detailed attribute information of each of the drugs, by preprocessing the first data set, standardizing a class included in the second data set and giving directionality, by preprocessing the second data set, extracting expressions representing a side effect type included in the third data set, normalizing the expressions and giving directionality to the third data set, by preprocessing the third data set, training at least one artificial intelligence model using the preprocessed first, second, and the third data set, and determining the grade and type of the side effect of a pair of drugs using the at least one artificial intelligence model.

### TECHNICAL FIELD

The present disclosure relates to drug-drug interaction (DDI) analysis and, more particularly, to a method and device for analyzing drug-drug interaction using an artificial intelligence algorithm.

### BACKGROUND

As civilization develops, human life has become richer, but new diseases continue to occur. In accordance with people's wishes to live long, healthy lives free from disease, numerous attempts and efforts are being made to create new drugs. Meanwhile, in the pharmaceutical industry, the fourth industrial revolution is presenting direction in drug development through artificial intelligence and bio-convergence technology based on large-scale data.

Drug-drug interaction (DDI) is one of the major considerations in drug development. DDI, in a broad sense, refer to a phenomenon that occurs when the efficacy or toxicity of one drug is modified by another drug, food, environmental chemicals, etc. In other words, in clinical practice, DDI means that two or more drugs affect each other when used together at the same time or at short intervals. According to the domestic combination drug clinical trial criterion, in terms of safety, the criterion ‘when considering the pathological mechanism and therapeutic mechanism, etc., in the case of drugs that are likely to exhibit pharmacokinetic and pharmacodynamic interactions when co-administered with individual main ingredients, a close evaluation for the safety and efficacy of combined administration is required’ is specified. In other words, when evaluating drug-drug interaction, both the direct effects of drugs on the body and the effects of reactions between drugs must be considered. However, since there are more than a thousand types of existing drugs, and the approval requirements for drug-drug interaction differ between countries and organizations, even the data that can be used to determine whether a combination of drugs is appropriate is incomplete.

### DISCLOSURE

### Technical Problem

The present disclosure is to provide a method and device for effectively analyzing drug-drug interaction (DDI) using an artificial intelligence algorithm.

The present disclosure is to provide a method and device for reducing consumption of time, human, and material resources required for analyzing drug-drug interaction using an artificial intelligence algorithm.

The present disclosure is to provide a method and device for quickly predicting appropriate drug combinations for combination therapy based on an artificial intelligence algorithm. The present disclosure is to provide a method and device for quickly recommending appropriate drug combinations for combination drugs based on an artificial intelligence algorithm.

The technical problem of the present disclosure relates to a video editing method, device, and computer program capable of performing backup, and provides a video editing method, device, and computer program capable of performing backup so that backup media of an edited video may be stored and utilized in subsequent editing.

### Technical Solution

According to the present disclosure, there is provided a method for analyzing drug-drug interaction, the method including: acquiring a first data set for chemical structures of drugs, a second data set for a grade of a side effect between the drugs and a third data set for a type of a side effect between the drugs, as data sets for training, at a processor configured to analyze the DDI; generating detailed attribute information of each of the drugs, by preprocessing the first data set, at the processor; standardizing a class included in the second data set and giving directionality, by preprocessing the second data set, at the processor; extracting expressions representing a side effect type included in the third data set, normalizing the expressions and giving directionality to the third data set, by preprocessing the third data set, at the processor; training at least one artificial intelligence model stored in a memory using the preprocessed first data set, the preprocessed second data set and the preprocessed third data set, at the processor; and determining the grade and type of the side effect of a pair of drugs from information on the pair of drugs using the at least one artificial intelligence model, at the processor.

According to the embodiment of the present disclosure in the method, the training the at least one artificial intelligence model may include generating a training data set mapping the grade and type of the side effect to an attribute combination of the drug, by matching the preprocessed first set data with the preprocessed second data set and the preprocessed third data set.

According to the embodiment of the present disclosure in the method, the determining the grade and type of the side effect between the pair of drugs from the information on the pair of drugs may include generating detailed attribute information of each of the pair of drugs, by preprocessing the information on the pair of drugs, at the processor; and inputting the detailed attribute information as input data of the at least one artificial intelligence model, at the processor.

According to the embodiment of the present disclosure in the method, the detailed attribute information may include BDSI (Binary data of Drug Structural Information), ISD (Index of Similarity between Drugs), IIPD (Index of Interaction between Protein and Drug), IISD (Index of Interaction Similarity between Drugs), and ADMET (Absorption Distribution Metabolism Excretion Toxicity) of each drug.

According to the embodiment of the present disclosure in the method, the second data set may include side effect grade data between first drugs collected from a first source and side effect grade data between second drugs from a second source. The side effect grade data between the first drugs and the side effect grade data between the second drugs may indicate the same class with different expressions. Also, the different expressions indicating the same class may be normalized through the preprocessing.

According to the embodiment of the present disclosure in the method, the third data set may include a first sentence expressing a type of a side effect of a first pair of drugs and a second sentence expressing a type of a side effect of a second pair of drugs. Each of the first sentence and the second sentence may include an expression indicating at least one type. Also, the first sentence and the second sentence may include different expressions indicating the type of the same meaning. The different expressions indicating the type of the same meaning may be replaced with a single term through the preprocessing.

According to the embodiment of the present disclosure in the method, the second data set may include an item including side effect grade information of a pair of drugs combined in order of a first drug and a second drug. The preprocessed second data set may be processed to further include side effect grade information of the pair of drugs combined in order of the second drug and the first drug by giving the directionality.

According to the embodiment of the present disclosure in the method, the third data set may include an item including side effect type information of a pair of drugs combined in order of a first drug and a second drug. Also, the preprocessed third data set may be processed to further include side effect type information of the pair of drugs combined in order of the second drug and the first drug by giving the directionality.

According to the embodiment of the present disclosure in the method, the at least one artificial intelligence model may include a first artificial intelligence model of multiple input and a single output predicting the side effect grade and a second artificial intelligence model of multiple input and multiple output predicting the side effect type.

According to the embodiment of the present disclosure in the method, the method may further include transmitting data indicating a grade and type of a side effect between the pair of drugs to another device.

According to another embodiment of the present disclosure, there is provided a device for analyzing drug-drug interaction, the device including: a memory configured to store at least one artificial intelligence model; and a processor connected to the memory. The processor is configured to: acquire a first data set for chemical structures of drugs, a second data set for a grade of a side effect between the drugs and a third data set for a type of a side effect between the drugs, as data sets for training; generate detailed attribute information of each of the drugs, by preprocessing the first data set; normalizing a class included in the second data set and giving directionality, by preprocessing the second data set; extract expressions representing a side effect type included in the third data set, normalizing the expressions and giving directionality to the third data set, by preprocessing the third data set; learn the at least one artificial intelligence model using the preprocessed first data set, the preprocessed second data set and the preprocessed third data set; and determine the grade and type of the side effect of a pair of drugs from information on the pair of drugs using the at least one artificial intelligence model.

The features briefly summarized above for this disclosure are only exemplary aspects of the detailed description of the disclosure which follow, and are not intended to limit the scope of the disclosure.

### Effects of Invention

According to the present disclosure, it is possible to reduce consumption of time, human, material resources required for drug-drug interaction (DDI) analysis.

It will be appreciated by persons skilled in the art that that the effects that can be achieved through the present disclosure are not limited to what has been particularly described hereinabove and other advantages of the present disclosure will be more clearly understood from the detailed description.

### DESCRIPTION OF DRAWINGS

FIG. 1 shows a structure of a drug-drug interaction analysis system according to an embodiment of the present disclosure.

FIG. 2 shows a structure of an artificial neural network applicable to a system according to an embodiment of the present disclosure.

FIG. 3 shows a structure of a system according to an embodiment of the present disclosure.

FIG. 4 shows the concept of acquiring training data and independent variables in a system according to an embodiment of the present disclosure.

FIG. 5 shows a functional structure of a system according to an embodiment of the present disclosure.

FIG. 6 shows an example of an operation for deriving BDSI (Binary data of Drug Structural Information) from a chemical structure according to an embodiment of the present disclosure.

FIG. 7 shows an example of an operation for deriving an ISD (Index of Similarity between Drugs) from a chemical structure according to an embodiment of the present disclosure.

FIG. 8 shows an example of an operation for matching attribute information and grade/type information according to an embodiment of the present disclosure.

FIG. 9 shows an example of training evaluating an artificial intelligence model according to an embodiment of the present disclosure.

FIGS. 10A and 10B show an artificial intelligence model for a side effect type system according to an embodiment of the present disclosure.

FIGS. 11A and 11B show an example of an artificial intelligence model for a side effect grade system according to an embodiment of the present disclosure.

FIG. 12 shows an example of an artificial intelligence model for a side effect grade system according to an embodiment of the present disclosure.

FIG. 13 shows a cyclical operation of prediction-verification-training of an artificial intelligence model according to an embodiment of the present disclosure.

FIG. 14 shows an example of an artificial intelligence model for a side effect type system according to an embodiment of the present disclosure.

FIG. 15 shows a procedure for analyzing drug-drug interaction in a system according to an embodiment of the present disclosure.

FIG. 16 shows an example of a procedure for training and prediction in a system according to an embodiment of the present disclosure.

FIG. 17 shows an example of a procedure for performing training in a system according to an embodiment of the present disclosure.

FIG. 18 shows an example of a procedure for performing prediction in a system according to an embodiment of the present disclosure.

### DETAILED DESCRIPTION

Hereinafter, with reference to the accompanying drawings, embodiments of the present disclosure will be described in detail so that those skilled in the art can easily practice them. However, the present disclosure may be implemented in many different forms and is not limited to the embodiments described herein.

In describing embodiments of the present disclosure, if it is determined that detailed descriptions of known configurations or functions may obscure the subject matter of the present disclosure, detailed descriptions thereof will be omitted. In addition, in the drawings, parts that are not related to the description of the present disclosure are omitted, and similar parts are given similar reference numerals.

The present disclosure proposes a technology for analyzing drug-drug interaction (DDI) using an intelligent artificial intelligence algorithm. Specifically, the present disclosure is to provide a system for analyzing drug-drug interaction in various environments such as cloud environments and local environments.

A system according to various embodiments of the present disclosure may be named ‘CombiRisk’. CombiRisk is a system that analyzes drug-drug interaction based on big data and artificial intelligence technology, which are core technologies of the fourth industry, and can quickly predict and recommend appropriate drug combinations for combination drugs to users. The CombiRisk system, based on domestic and foreign drug big data and deep training technology, is a decision support system that predicts suitability between the main ingredients of drugs, and is designed to help arrange time, human, and material resources for combination drug research more efficiently.

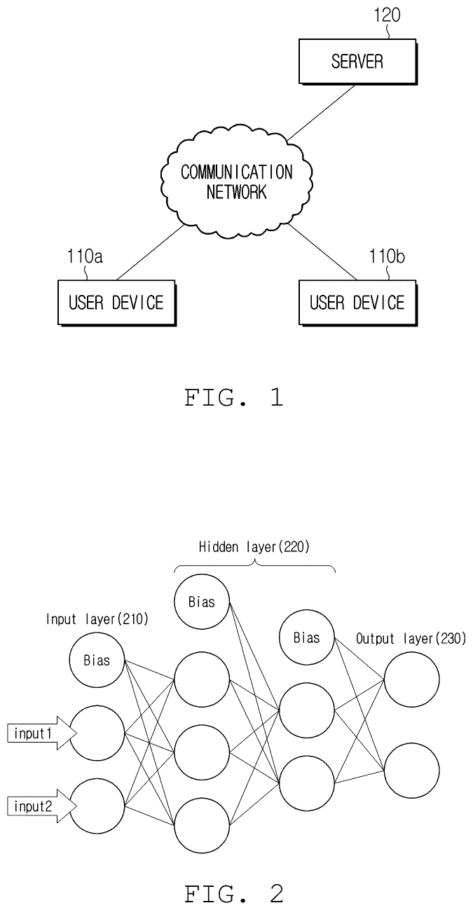

FIG. 1 shows a structure of a drug-drug interaction analysis system according to an embodiment of the present disclosure.

Referring to FIG. 1, the system includes a user device 110a, a user device 110b, and a server 120 connected to a communication network. FIG. 1 illustrates two user devices 110a and 110b, but there may be three or more user devices.

The user devices 110a and 110b are terminal devices used by a user who wishes to perform drug-drug interaction analysis using the system according to the embodiment of the present disclosure. The user devices 110a and 110b may acquire input data (e.g., information on drugs that are the subject of interaction analysis) and transmit the input data to the server 120 through a communication network. Each of the user devices 110a and 110b may include a communication unit for communication, a storage unit for storing data and programs, a display unit for displaying information, an input unit for user input, and a processor for control. For example, each of the user devices 110a and 110b may be a general-purpose device (e.g., smartphone, tablet, laptop computer, desktop computer) or a system-specific access terminal in which an application or program for system access is installed.

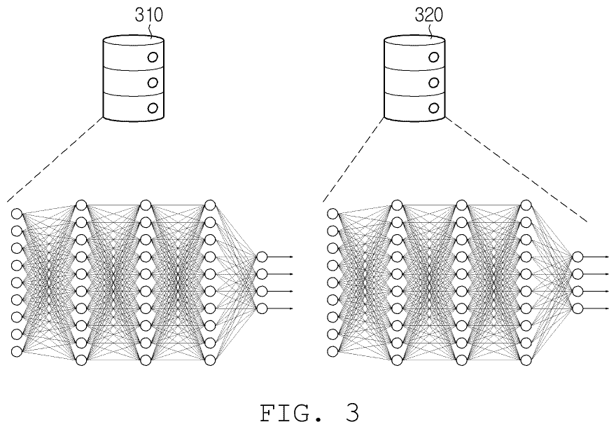

The server 120 performs calculation to analyze drug-drug interaction according to embodiments of the present disclosure. The server 120 provides various functions for the drug-drug interaction analysis system and can operate an artificial intelligence model. An example of an artificial neural network applicable to the present disclosure will be described below with reference to FIG. 2. In addition, the server 120 may perform training for an artificial intelligence model using training data. Here, the server 120 may be a local server existing in a local network or a remote access server (e.g., cloud server) connected through an external network. The server 120 may include a communication unit for communication, a storage unit for storing data and programs, and a processor for control.

FIG. 2 shows a structure of an artificial neural network applicable to a system according to an embodiment of the present disclosure. The artificial neural network shown in FIG. 2 may be understood as the structure of artificial intelligence models stored in the server 120. Referring to FIG. 2, the artificial neural network includes an input layer 210, at least one hidden layer 220, and an output layer 230. Each of the layers 210, 220, and 230 is composed of a plurality of nodes, and each of the nodes is connected to the output of at least one node belonging to a previous layer. Each node adds a bias to an inner product of each output value of the nodes in the previous layer and a connection weight corresponding thereto, and then forwards an output value multiplied by a non-linear activation function to at least one neuron of a next layer. Each layer may be further divided into input node, perceptron, and output node.

The artificial neural network shown in FIG. 2 may be formed through training (e.g., machine learning, deep learning, etc.). In addition, the artificial neural network models used in various embodiments of the present disclosure include at least one of a fully convolutional neural network, a convolutional neural network, a recurrent neural network, and a restricted Boltzmann machine (RBM) or a deep belief neural network (DBN), but is not limited thereto. Alternatively, machine learning methods other than deep learning may also be included. Alternatively, a hybrid model which is a combination of deep learning and machine learning may also be included. For example, a deep learning-based model may be applied to extract features of an image, and a machine learning-based model may be applied when classifying or recognizing the image based on the extracted features. The machine learning-based model may include Support Vector Machine (SVM), AdaBoost, etc., but are not limited thereto.

For deep learning of the system according to an embodiment of the present disclosure, a deep neural network (DNN) may be applied, and drug-drug interaction that may appear on pharmacokinetics (PK) may be predicted. That is, the system according to the embodiment of the present disclosure applies deep learning technology to drug-drug interaction information, predicts the risk level and type of side effects due to interaction between new drugs, and provides predicted results. To this end, after collecting DDI information provided at home and abroad, a database may be built, and the features of each drug may be extracted based on this. For example, the features of the drug may include drug structure information, structural similarity between drugs, absorption/distribution/metabolism/excretion/toxicity information (ADMET), and interaction information with proteins. Here, ADMET is information used as a criterion to describe the distribution of a drug in a body.

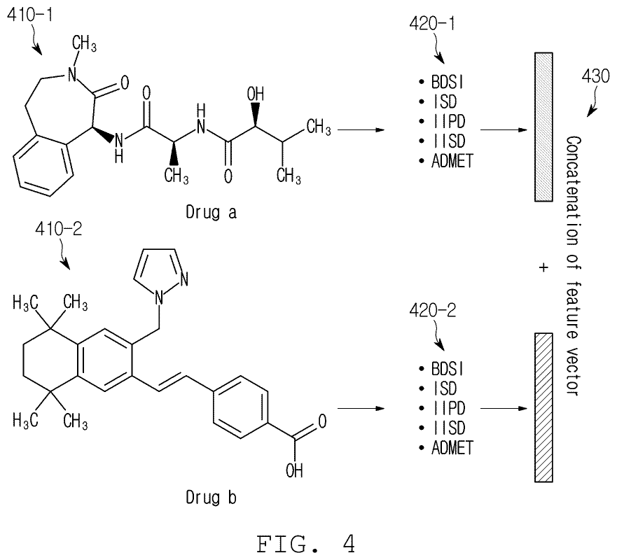

The system according to the embodiment of the present disclosure may be composed of a DDI grade system that predicts the risk in 5 levels and a DDI type system that predicts what type of DDI will occur, as shown in FIG. 3 below.

FIG. 3 shows a structure of a system according to an embodiment of the present disclosure. Referring to FIG. 3, the server 120 includes a DDI type system 310 and a DDI grade system 320. The DDI type system 310 may be implemented based on a dragbank database. The DDI grade system 320 may be implemented based on the drugbank database, drugscom database, a Health Insurance Review and Assessment Service complex prescription database, a drug combination database, a combination contraindication database, etc.

The DDI type system 310 provides the type of DDI that is expected to occur. For example, the DDI type system 310 may predict that the risk or severity of a specific symptom (e.g., rhabdomyolysis) may increase when drug a, which is subject drug, and drug b, which is affecting drug, are combined. The DDI type system 310 may be referred to as ‘RiskDescription system’, ‘RiskDescription system model’, ‘CombiType system’, ‘CombiType system model’, ‘Side effect type system’, etc.

The DDI grade system 320 may include a DNN for predicting risk. The DDI grade system 320 may be referred to as ‘RiskGrade system’, ‘RiskGrade system model’, ‘CombiGrade system’, ‘CombiGrade system model’, ‘Side effect grade system’, etc. According to one embodiment of the present disclosure, the risk may be classified into five levels. For example, the risks classified into five levels are shown in [Table 1] below.

TABLE 1
5-level risk

| | |
|---|---|
| Level 1 | contraindicated |
| Level 2 | major |
| Level 3 | moderate |
| Level 4 | minor |
| Level 5 | available |

FIG. 4 shows the concept of acquiring training data and independent variables in a system according to an embodiment of the present disclosure. Referring to FIG. 4, training data and independent variables may be acquired from the chemical structure of the drug. By analyzing the chemical structure information of drug a 410-1 and drug b 410-2, detailed attribute information 420-1 and 420-2 of the drug a 410-1 and the drug b 410-2 may be acquired. For example, the detailed attribute information 420-1 and 420-2 may include BDSI, ISD, IIPD, IISD, ADMET, etc. Thereafter, a concatenation 430 of a feature vector is generated based on the attribute information 420-1 and 420-2. The concatenation 430 of the feature vector may be used for training or prediction operations of an artificial intelligence model.

In various embodiments of the present disclosure, the drug-drug interaction prediction system integratively uses a plurality of DDI databases (e.g., Drugbank, Drugscom, Public Data Portal, Health Insurance Review and Assessment Service, Korea Institute of Drug Safety and Risk Management, etc.). Therefore, as the artificial intelligence model is learned using a variety of data without being biased toward one type of database, the risk of overfitting is significantly lowered. In addition, the drug-drug interaction prediction system according to various embodiments of the present disclosure may not only predict the results of the drug-drug interaction, but may also simultaneously predict the severity of the drug-drug interaction. Accordingly, resources needed for combination drug development and drug prescription may be utilized more efficiently. In addition, the drug-drug interaction prediction system according to various embodiments of the present disclosure considers not only the reaction of the drug-drug interaction but also the directionality of the interaction between two drugs to provide prediction for distinguishing between the subject drug and the affected drug.

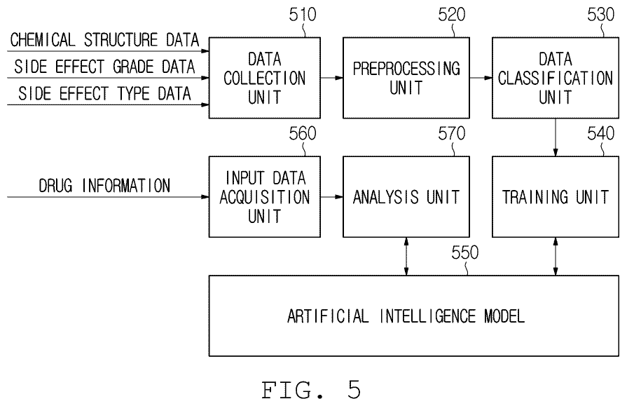

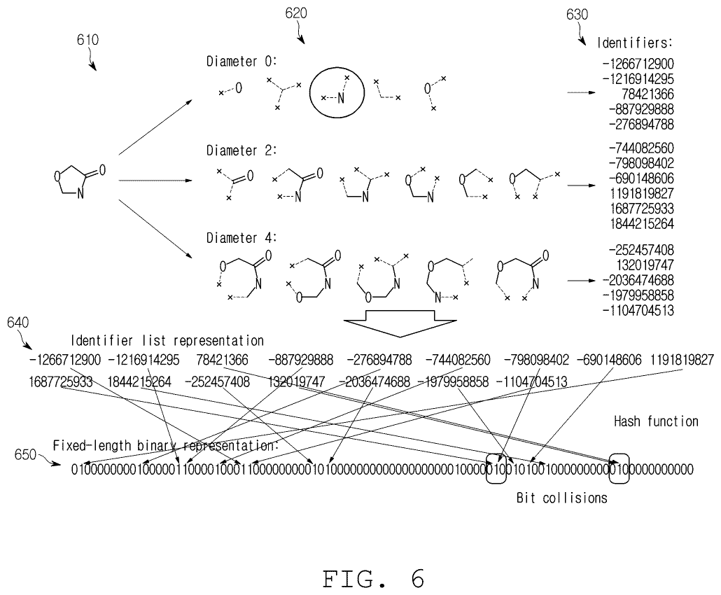

FIG. 5 shows a functional structure of a system according to an embodiment of the present disclosure. FIG. 5 may be understood as the functional configuration of the server 120 of FIG. 1.

Referring to FIG. 5, the server includes a data collection unit 510, a preprocessing unit 520, a data classification unit 530, a training unit 540, an artificial intelligence model 550, an input data acquisition unit 560, and an analysis unit 570.

The data collection unit 510 collects original data for training. The original data may include data of various forms and contents. For example, the original data may include paid purchase data and public data. According to one embodiment, the original data may include three data sets. Specifically, the original data may include a drug chemical structure data set, a side effect grade data set between drugs, and a side effect type data set between drugs.

The preprocessing unit 520 processes the original data for training. In other words, the preprocessing unit 520 processes the original data into a learnable form. According to various embodiments, the preprocessing unit 520 may generate detailed attribute information indicating the attributes of the drug from the drug structure data set. For example, the detailed attribute information may include Binary data of Drug Structural Information (BDSI), Index of Similarity between Drugs (ISD), Index of Interaction between Protein and Drug (IIPD), Index of Interaction Similarity between Drugs (IISD), and Absorption Distribution Metabolism Excretion Toxicity (ADMET). According to various embodiments of the present disclosure, the detailed attribute information may further include items other than BDSI, ISD, IIPD, IISD, and ADMET listed above, or at least one of the listed items may be replaced with another item. In addition, the preprocessing unit 520 may normalize the side effect grade data set between drugs and the side effect type data set between drugs according to a predefined criterion, give a directionality, match them with the detailed attribute information of the drug, and then generate an independent variable and a dependent variable.

The data classification unit 530 classifies the preprocessed data according to its use in the training procedure of the artificial intelligence model 550. For example, the data classification unit 530 may classify data into training data, verification data, and test data. Specifically, 60% of the preprocessed data may be classified as training data, 20% as verification data, and 20% as test data.

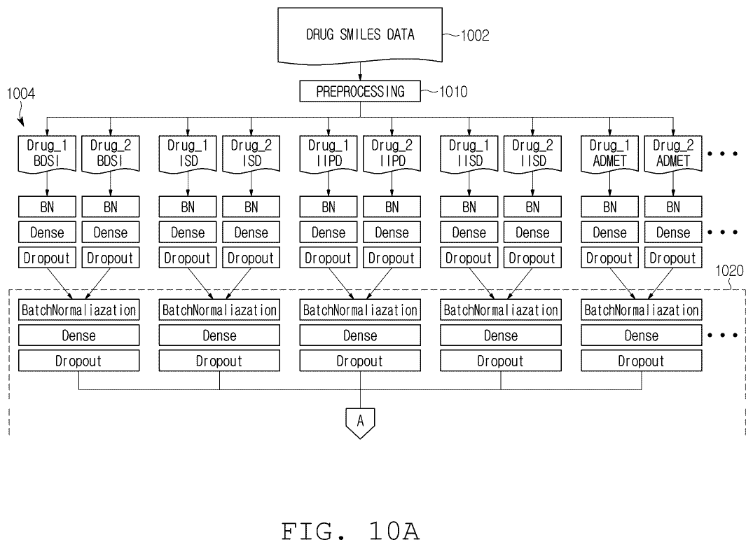

The training unit 540 performs training and evaluation of the artificial intelligence model 550 using the training data, verification n data, and test data provided from the data classification unit 530. For example, as shown in FIG. 9, the artificial intelligence model 550 may be learned and evaluated. Referring to FIG. 9, after the artificial intelligence model 550 is learned using the training data 910 and the verification data 920, performance is evaluated using the test data 930. At this time, if performance does not meet a required criterion (e.g., accuracy greater than or equal to a critical ratio), re-training may be performed. In consideration of re-training, the training unit 540 performs training using only part of the training data and verification data, performs evaluation using part of the test data, and then additionally performs training using another part according to the evaluation results.

The artificial intelligence model 550 includes a deep neural network. As explained with reference to FIG. 2, the deep neural network includes an input layer, an output layer, and at least one hidden layer. Each layer consists of at least one input node, at least one perceptron, and at least one output node. According to an embodiment of the present disclosure, the deep neural network may be quickly built by building a neural layer using an artificial intelligence development library using Python-based Keras library, PyTorch library, etc. For example, Python-based Keras library, Pytorch library, tensorflow library, etc. may be used, or other programming languages (e.g. JAVA, C, etc.) may be used.

Each of the DDI type system 310 and the DDI grade system 320 included in the CombiRisk system may be designed to include approximately 6 to 8 layers. At this time, each layer may include a batch normalization layer, a dense layer, and a dropout layer. Through the batch normalization layer, the data may be converted into a better state for training, training may be performed in the dense layer, and the probability of overfitting may be reduced in the dropout layer. The number of perceptrons (neurons) in each layer may be designed to be between a minimum of 15 and a maximum of 2048. It is desirable to apply and test several algorithms to resolve imbalance between classes in the training data. For example, a focal loss algorithm that lowers the loss value of a well-predicted class and slightly lowers the loss value of a poorly-predicted class may be applied. In this case, training is performed more intensively on the poorly-predicted class.

The input data acquisition unit 560 acquires input data input to the artificial intelligence model 550 for prediction operation. For example, the input data includes drug information. Here, the drug information may include detailed information about the attributes of the drug (e.g., BDSI, ISD, IIPD, IISD, ADMET, etc.). Alternatively, the drug information may include a drug ID. When the input data is a drug ID, although not shown in FIG. 5, the preprocessing unit that generates detailed attribute information by preprocessing the input data may be further included. Alternatively, the preprocessing unit 520, which preprocesses the training data, may preprocess the input data.

The analysis unit 570 inputs detailed attribute information included in the input data or detailed attribute information generated from the input data to the artificial intelligence model 550, acquires output data of the artificial intelligence model 550, and generates an analysis result based on the acquired output data. The generated analysis result may be stored internally or transmitted to the outside (e.g., the user device 110a or the user device 110b).

According to an embodiment of the present disclosure, the drug chemical structure data set may include a drug identifier (ID) and Simplified Molecular Input Line Entry System (SMILES). Here, SMILES is a string representing the structure of a chemical substance. SMILES follows a string notation method with a very concise structure and is a compressed abstract expression of the structural features of a compound. According to SMILES, atoms are represented by standard element symbols, hydrogen atoms are omitted because they are assumed to be connected wherever possible, neighboring atoms are written immediately adjacent, double bonds are written as ‘=’, and triple bonds are written as ‘#’, bond branches are expressed with parentheses ‘( )’, and ring structures are expressed by numbering the atoms connected to each other. For example, the SMILES representation of ethanol is CCO, the SMILES representation of benzene is C1=CC═CC═C1, and the SMILES representation of anthracene is C1=CC═C2C═C3C═CC═CC3=CC2=C1. SMILES of each compound may be acquired from chemical databases such as Pubchem and Drugbank. However, since there are differences in the SMILES format for each database, preprocessing may be necessary. However, the above-described SMILES is an example of a chemical structure data set, and other chemical structure data may be used for various embodiments. For example, in place of or in parallel with SMILES, compound data (mol file, mol2 file, sdf (structural-data file)), InChI (International Chemical Identifier), chemical formula, 3D structure information, etc. may be used. According to an embodiment of the present disclosure, the side effect grade data set between drugs includes a first drug ID, a second drug ID, and a grade value indicating the degree of side effects. The grade value is one of predefined candidate values, and each candidate value indicates one of the levels listed in [Table 1]. The side effect type data set between drugs includes a subject first drug ID, an affected second drug ID, and type information indicating the side effect type. The type information may be expressed as a sentence describing what side effects the first drug causes to the second drug. For example, the type information may be expressed as “sub-dug may decrease the anticoagulant activities of aff_drug”, “sub-dug may decrease the antihypertensive activities of aff_drug”, “sub-dug can cause a decrease in the absorption of aff_drug”, “sub-dug can cause an increase in the absorption of aff_drug”, etc.

According to an embodiment of the present disclosure, the side effect grade data set between drugs and the side effect type data set between drugs are related to a combination of drugs included in the drug structure data set, and thus include more items than the items included in the drug structure data set. For example, if the drug structure data set includes the structure information of about 13,000 drug items, each of the side effect grade data set between drugs and the side effect type data set between drugs may include about 1,500,000 interaction-related items.

In the embodiment described with reference to FIG. 5, the preprocessing unit 520 generates detailed attribute information including BDSI, ISD, IIPD, IISD, and ADMET from the chemical structure of the drug. A description of each attribute is as follows.

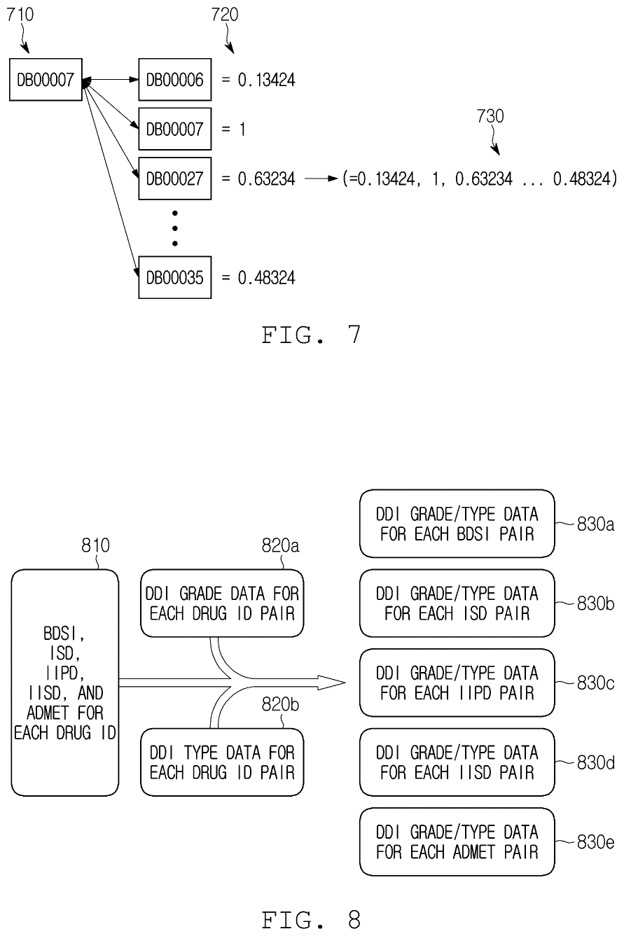

BDSI represents unique information of compounds and is information designed to confirm the features or similarity of molecules in drugs. The preprocessing unit 520 calculates each value according to which structure it has or which element it is coupled with depending on a distance from each element, and expresses the calculated values as binary values. In other words, BDSI uses surrounding elements to represent the average molecular structure and the features of various molecules. An example of the process of deriving BDSI from the chemical structure is shown in FIG. 6 below.

FIG. 6 shows an example of an operation for deriving BDSI from a chemical structure according to an embodiment of the present disclosure. FIG. 6 illustrates generation of BDSI for a compound 610. Referring to FIG. 6, for each element of the compound 610, structures 620 in distance ranges of 0, 2, and 4 are identified. The identified structures 620 are converted into IDs 630 expressed numerically. The IDs 630 are converted into a list representation 640, and the binary values corresponding to the IDs included in the list representation 640 are rearranged by a hash function, thereby generating the BDSI 650.

The number of binaries to express DBSI may be adjusted, and since it is binary data, it has the advantage of being able to perform fast operations. An example of the generated BSDI data set is shown in [Table 2] below.

TABLE 2

| drug ID | BDSI |
|---|---|
| DB00006 | {1, 0, 0, 0, 0 . . . , 0, 1, 0, 1, 0} |
| DB00007 | {0, 0, 0, 0, 0 . . . , 0, 0, 0, 1, 0} |
| DB00014 | {1, 0, 0, 1, 0 . . . , 0, 0, 0, 0, 0} |
| DB00027 | {0, 0, 0, 0, 0 . . . , 0, 1, 0, 0, 0} |
| DB00035 | {0, 0, 1, 0, 0 . . . , 0, 0, 0, 1, 0} |
| DB00050 | {1, 0, 0, 0, 1 . . . , 0, 1, 0, 0, 0} |
| . . . | . . . |

ISD is generated based on BDSI and expresses the structural similarity of the compound. That is, the preprocessing unit 520 calculates the similarity of compound structures between drugs based on the molecular structure expressed by BDSI. One ISD value is generated per drug. For example, if there are 10,000 drugs, there are 10,000 similarity values per drug, as shown in FIG. 7 below, and the 10,000 values arranged in order constitute one ISD. FIG. 7 shows an example of an operation for deriving an ISD from a chemical structure according to an embodiment of the present disclosure. Referring to FIG. 7, for a drug 710 with an ID of DB00007, similarity values 720 with all drugs, including itself, are calculated. The ISD value 730 is generated by arranging the similarity values 730 in a predefined order (e.g., drug ID ascending order). An example of the ISD data set generated through this process is shown in [Table 3] below.

TABLE 3

| drug ID | ISD |
|---|---|
| DB00006 | {1, 0.34234, 0.64534 . . . 0.756454} |
| DB00007 | {0.34234, 1, 0.342425 . . . 0.123546} |
| DB00014 | {0.64534, 0.342425, 1 . . . 0.856523} |
| DB00027 | {0.133345, 0.623244, 0.136542 . . . 0.643534} |
| DB00035 | {0.845634, 0.234562, 0.734211 . . . 0.892344} |
| DB00050 | {0.522423, 0.642324, 0.718964 . . . 0.716342} |
| . . . | . . . |

IIPD is information expressing a reaction between drug and protein. The preprocessing unit 520 quantifies a series of interactions that occur between drugs and proteins based on the molecular structure expressed by BDSI. To this end, proteins known to play a major role in drug-protein interaction are selected. The preprocessing unit 520 may extract information about a total of eight types of interactions depending on the drug and protein structures. For example, the eight types of interactions may include hydrophobic contacts, aromatic face to face, aromatic edge to face, and hydrogen bond (protein as hydrogen bond donor), hydrogen bond (protein as hydrogen bond acceptor), salt bridges (protein positively charged), salt bridges (protein negatively charged), salt bridges (ionic bond with metal ion), etc. IISD is information indicating the similarity of IIPD between drugs. The preprocessing unit 520 calculates the similarity of IIPD between drugs based on the IIPD. For example, if there are 10,000 drugs, there are 10,000 similarity values per drug, and a result of arranging 10,000 similarity values in order constitutes IISD of one drug. The preprocessing unit 520 determines an IISD for each drug and generates an IISD data set containing a plurality of IISDs.

ADMET is information that quantifies the grades of drug absorption, distribution, metabolism, excretion, and toxicity through changes in drug concentration in the body over time from a pharmacokinetics perspective. The preprocessing unit 520 extracts the molecular features of the drug from drug structure information, that is, SMILE, and then calculates ADMET. A total of 28 ADMET values representing each of the 6 categories per drug are generated. For example, the 28 ADMET values includes \textcircled{1} Log S, LodD, Log P related to basic physical and chemical properties, \textcircled{2} Caco-2, Pgp-Inhibitor, HIA, F(20%), F(30%) related to absorption, and \textcircled{3} PPB, VD, BBB related to distribution, \textcircled{4}CYP1A2-Inhibitor, CYP 1A2-Substrate, CYP 3A4-Inhibitor, CYP 3A4-Substrate, CYP 2C9-Inhibitor, CYP 2C9-Substrate, CYP 2C19-Inhibitor, CYP 2C19-Substrate, CYP 2D6-Inhibitor, CYP 2D6-Substrate related to metabolism, \textcircled{5} Clearance, T1/2 related to excretion, \textcircled{6} hERG, H-HT, Ames, Skin sensitivity, LD50 related to toxicity. The preprocessing unit 520 determines an ADMET value set for each drug and generates an IISD data set including a plurality of ADMET value sets.

The preprocessing unit 520 generates training data for the DDI grade system 320 from the side effect grade data set between drugs. There are original databases with classes divided into ‘0’ (Major), ‘1’ (Moderate), and ‘2’ (Minor), but there are also original databases with classes divided by other expressions or original databases without class divisions. Accordingly, the preprocessing unit 520 may analyze database characteristics for class reclassification and perform preprocessing based on the analysis result. In addition, the preprocessing unit 520 may give directionality to data. For example, if the item “DB06605+DB00001→Grade 1” is stored, the preprocessing unit 520 adds the item “DB00001+DB06605→Grade 1”. This is because, when training only the data of “DB06605+DB00001→Grade 1”, a result other than Grade 1 may be predicted if the combination of “DB00001+DB06605” is input.

The preprocessing unit 520 generates training data for the DDI type system 310 from the side effect type data set between drugs. The preprocessing unit 520 extracts the side effect type from the type information included in the side effect type data set between drugs. For example, when the type information is “sub_drug may decrease effectiveness of aff_drug”, the preprocessing unit 520 may extract ‘decrease’ and ‘effectiveness’. As another example, when the type information is “sub_drug may increase the QTc-prolonging activities of aff_drug”, the preprocessing unit 520 may extract ‘increase’ and ‘QTc-prolonging’. Then, the preprocessing unit 520 analyzes the type of the side effect and performs preprocessing. For example, the preprocessing unit 520 may organize synonyms, similar side effects, etc. into unified terms. In addition, the preprocessing unit 520 may give directionality to data. For example, if there is an item “DB06605+DB00001→increase, QTc-prolonging”, the preprocessing unit 520 may give a directionality value of ‘0’, meaning that DB06605 is an affected drug sub_drug and DB0001 is a subject drug aff_drug. In addition, the preprocessing unit 520 adds the item “DB00001+DB06605→increase, QTc-prolonging”, and give the directionality value of ‘1’, meaning that DB0001 is a subject drug aff_drug and DB06605 is an affected drug sub_drug. That is, the directionality value of ‘0’ means a combination in which the affected drug is the former and the subject drug is the latter, and the directionality value of ‘1’ means a combination in which the subject drug is the former and the affected drug is the latter.

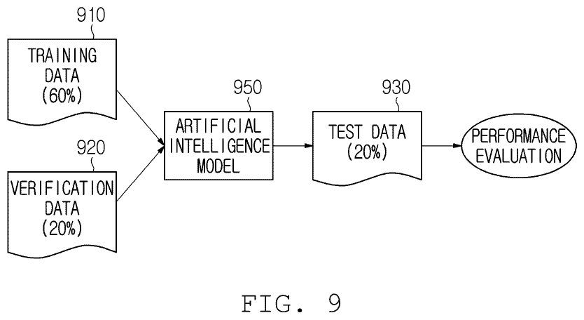

As described above, the preprocessing unit 520 performs preprocessing on the original data. Thereafter, the data is separated into independent and dependent variables. To determine the independent and dependent variables, the preprocessing unit 520 may match detailed attribute information and grade/type data such as BDSI, ISD, IIPD, IISD, ADMET, etc. from the chemical structure of the drug. The matching operation is shown in FIG. 8.

FIG. 8 shows an example of an operation for matching attribute information and grade/type information according to an embodiment of the present disclosure. Referring to FIG. 8, first, detailed attribute information 810 for each drug ID including BDSI, ISD, IIPD, IISD, and ADMET for each drug ID generated from chemical structure information (e.g., SMILES) is generated. Next, DDI grade data for each drug ID pair 820a and DDI type data for each drug ID pair 820b are matched with detailed attribute information for each drug ID 810. By matching, DDI grade/type data for each BDSI pair 830a, DDI grade/type data for each ISD pair 830b, DDI grade/type data for each IIPD pair 830c, DDI grade/type data for each IISD pair 830d, DDI grade/type data for each ADMET pair 830e are generated. For example, the DDI grade/type data 830a for each BDSI pair is shown in [Table 4] and [Table 5] below.

TABLE 4

| subject drug ID | affected drug ID | label |
|---|---|---|
| {1, 0, 0, 0, 0, . . . , 0, 1, 0, 1, 0} | {1, 0, 0, 1, 0, . . . , 0, 0, 0, 0, 0} | 0 |
| {1, 0, 0, 0, 0, . . . , 0, 1, 0, 1, 0} | {0, 0, 1, 0, 0, . . . , 0, 0, 0, 1, 0} | 0 |
| {0, 0, 1, 0, 0, . . . , 0, 0, 0, 1, 0} | {1, 0, 0, 1, 0, . . . , 0, 0, 0, 0, 0} | 1 |
| {0, 0, 1, 0, 0, . . . , 0, 0, 0, 1, 0} | {1, 0, 0, 0, 1, . . . , 0, 1, 0, 0, 0} | 1 |
| {1, 0, 0, 0, 0, . . . , 0, 1, 0, 1, 0} | {1, 0, 0, 0, 1, . . . , 0, 1, 0, 0, 0} | 2 |
| . . . | . . . | . . . |

TABLE 5

| subject drug ID | affected drug ID | modified summary |
|---|---|---|
| {1, 0, 0, 0, 0, . . . , 0, 1, 0, 1, 0} | {1, 0, 0, 1, 0, . . . , 0, 0, 0, 0, 0} | The therapeutic |
| | | efficacy |
| {1, 0, 0, 0, 0, . . . , 0, 1, 0, 1, 0} | {0, 0, 1, 0, 0, . . . , 0, 0, 0, 1, 0} | The therapeutic |
| | | efficacy |
| {0, 0, 1, 0, 0, . . . , 0, 0, 0, 1, 0} | {1, 0, 0, 1, 0, . . . , 0, 0, 0, 0, 0} | subject drug can |
| | | cause |
| {0, 0, 1, 0, 0, . . . , 0, 0, 0, 1, 0} | {1, 0, 0, 0, 1, . . . , 0, 1, 0, 0, 0} | subject drug can |
| | | cause |
| {1, 0, 0, 0, 0, . . . , 0, 1, 0, 1, 0} | {1, 0, 0, 0, 1, . . . , 0, 1, 0, 0, 0} | subject drug may |
| | | decrease |
| . . . | . . . | . . . |

The DDI grade/type data for each ISD pair 830b, the DDI grade/type data for each IIPD pair 830c, the DDI grade/type data for each IISD pair 830d, and the DDI grade/type data for each ADMET pair 830e may be constructed in the form similar to [Table 4] and [Table 5]. That is, by replacing the drug ID column in the grade/type data with the ISD, IIPD, IISD, and ADMET of the corresponding drug, the DDI grade/type data for each ISD pair 830b, the DDI grade/type data for each IIPD pair 830c, the DDI grade/type data for each IISD pair 830d, and the DDI grade/type data for each ADMET pair 830e may be generated. The preprocessing unit 520 determines independent and dependent variables from the data sets generated through matching. For example, independent variables such as BDSI, ISD, IIPD, IISD, and ADMET for drug pair may be generated as shown in [Table 6] to [Table 10] below.

TABLE 6

| subject drug ID | affected drug ID |
|---|---|
| DB06605_BDSI | DB00001_BDSI |
| DB06695_BDSI | DB00001_BDSI |
| DB01254_BDSI | DB00001_BDSI |
| DB00001_BDSI | DB01609_BDSI |
| DB00001_BDSI | DB01586_BDSI |
| DB00001_BDSI | DB02659_BDSI |
| . . . | . . . |

In [Table 6], ‘DB*****_DBSI’ means the BDSI value of the drug whose drug ID is DB*****.

TABLE 7

| subject drug ID | affected drug ID |
|---|---|
| DB06605_ISD | DB00001_ISD |
| DB06695_ISD | DB00001_ISD |
| DB01254_ISD | DB00001_ISD |
| DB00001_ISD | DB01609_ISD |
| DB00001_ISD | DB01586_ISD |
| DB00001_ISD | DB02659_ISD |
| . . . | . . . |

In [Table 7], ‘DB*****_ISD’ means the ISD value of the drug whose drug ID is DB*****.

TABLE 8

| subject drug ID | affected drug ID |
|---|---|
| DB06605_IIPD | DB00001_IIPD |
| DB06695_IIPD | DB00001_IIPD |
| DB01254_IIPD | DB00001_IIPD |
| DB00001_IIPD | DB01609_IIPD |
| DB00001_IIPD | DB01586_IIPD |
| DB00001_IIPD | DB02659_IIPD |
| . . . | . . . |

In [Table 8], ‘DB*****_IIPD’ means the IIPD value of the drug whose drug ID is DB*****.

TABLE 9

| subject drug ID | affected drug ID |
|---|---|
| DB06605_IISD | DB00001_IISD |
| DB06695_IISD | DB00001_IISD |
| DB01254_IISD | DB00001_IISD |
| DB00001_IISD | DB01609_IISD |
| DB00001_IISD | DB01586_IISD |
| DB00001_IISD | DB02659_IISD |
| . . . | . . . |

In [Table 9], ‘DB*****_IISD’ means the IISD value of the drug whose drug ID is DB*****.

TABLE 10

| subject drug ID | affected drug ID |
|---|---|
| DB06605_ADMET | DB00001_ADMET |
| DB06695_ADMET | DB00001_ADMET |
| DB01254_ADMET | DB00001_ADMET |
| DB00001_ADMET | DB01609_ADMET |
| DB00001_ADMET | DB01586_ADMET |
| DB00001_ADMET | DB02659_ADMET |
| . . . | . . . |

In [Table 10], ‘DB*****_ADMET’ means the ADMET value of the drug whose drug ID is DB*****. In addition, items for grade and type are extracted as dependent variables. For example, dependent variables that include a grade class of a single output shown in [Table 11] below, and a type class of multiple outputs shown in [Table 12] below may be generated.

TABLE 11
label

0
0
1
1
2
2
. . .

TABLE 12

| QTc- | | liver | | | | | | | |
|---|---|---|---|---|---|---|---|---|---|
| Prolonging | hepatotoxic | damage | infection | hypothyroid | hypomania | hyperthemia | hyperkalemic | hypertension | . . . |
| 0 | 0 | 1 | 0 | 0 | 0 | 1 | 0 | 0 | . . . |
| 0 | 0 | 1 | 0 | 0 | 1 | 0 | 0 | 0 | . . . |
| 0 | 0 | 0 | 0 | 0 | 0 | 0 | 0 | 1 | . . . |
| 0 | 0 | 0 | 0 | 1 | 0 | 0 | 0 | 0 | . . . |
| 1 | 0 | 0 | 0 | 0 | 0 | 1 | 0 | 0 | . . . |
| 1 | 0 | 0 | 0 | 0 | 0 | 0 | 0 | 1 | . . . |
| . . . | . . . | . . . | . . . | . . . | . . . | . . . | . . . | . . . | . . . |

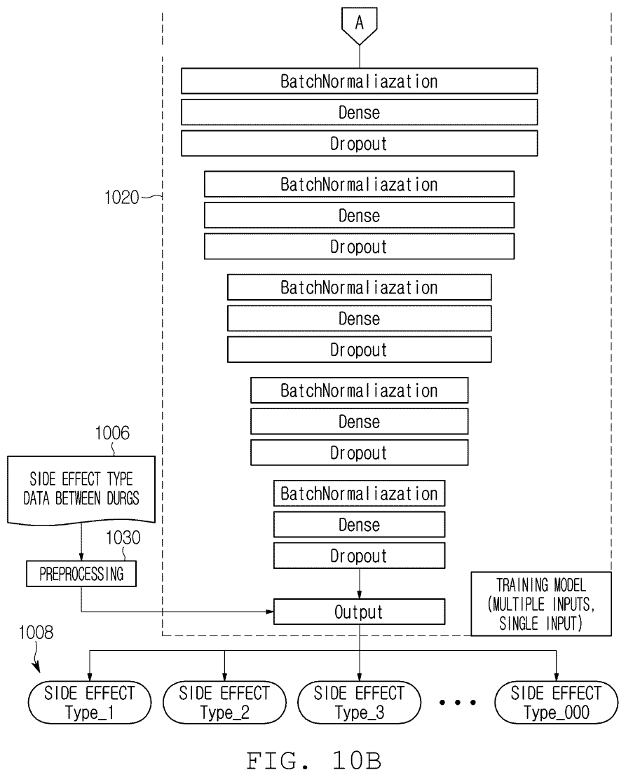

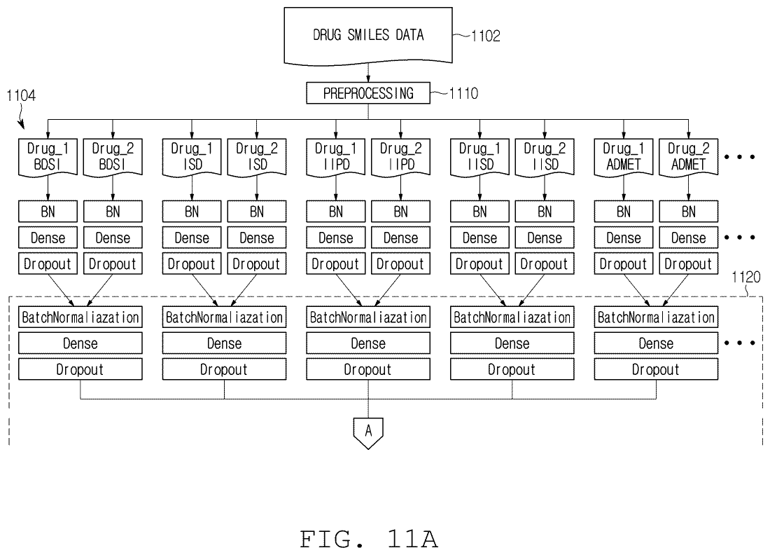

FIGS. 10A and 10B show an example of an artificial intelligence model for a side effect type system according to an embodiment of the present disclosure. FIGS. 10A and 10B show an artificial intelligence model for a DDI type system 310. Referring to FIGS. 10A and 10B, detailed attribute information 1004 including BDSI, ISD, IIPD, IISD, and ADMET is determined from drug SMILES data 1002 through a preprocessing process 1010. The detailed attribute information 1004 is provided as training data to the artificial intelligence model 1020. The artificial intelligence model 1020 includes a plurality of layers, and each layer includes a batch normalization (BN) layer, a dense layer, and a dropout layer. The side effect type 1008 is determined by prediction using the artificial intelligence model 1020. The side effect type 1008 takes the form of multiple outputs. The drug-to-drug side effect type data 1006 is subjected to a pre-processing process 1030 and is then provided to the output layer of the artificial intelligence model 1020, and the artificial intelligence model 1020 is learned through a back-propagation operation. Here, the structure of the output layer may depend on the type of dependent variable.

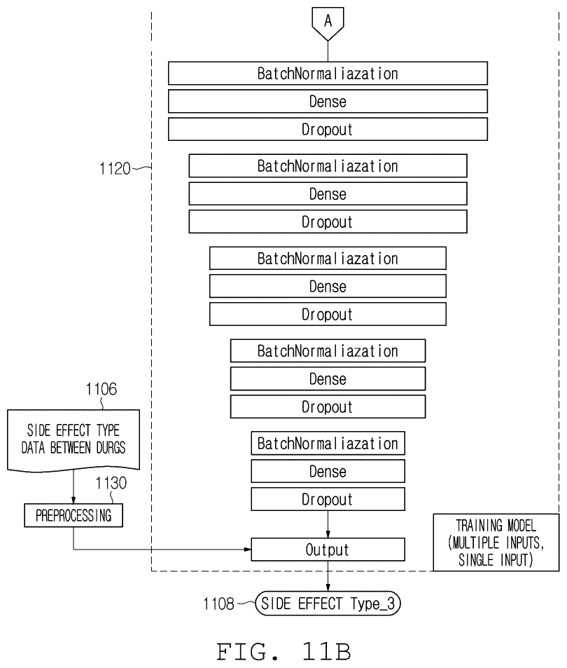

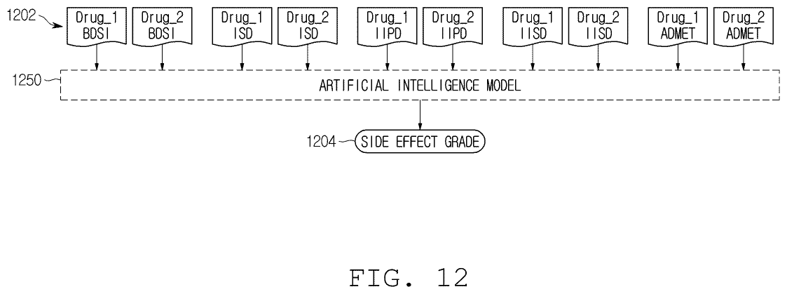

FIGS. 11A and 11B show an example of an artificial intelligence model for a side effect grade system according to an embodiment of the present disclosure. FIGS. 11A and 11B illustrate an artificial intelligence model for the DDI grade system 320. Referring to FIG. 11, detailed attribute information 1104 including BDSI, ISD, IIPD, IISD, and ADMET is determined from drug SMILES data 1102 through a preprocessing process 1110. The detailed attribute information 1104 is provided as training data to an artificial intelligence model 1120. The artificial intelligence model 1120 includes a plurality of layers, and each layer includes a batch normalization (BN) layer, a dense layer, and a dropout layer. The side effect grade 1108 is determined by prediction using the artificial intelligence model 1120. The side effect grade 1108 takes the form of a single output. The side effect grade data 1106 between drugs is subjected to a preprocessing process 1130 and is then provided to the output layer of the artificial intelligence model 1120, and the artificial intelligence model 1120 is learned through a back-propagation operation. Here, the structure of the output layer may depend on the type of dependent variable.

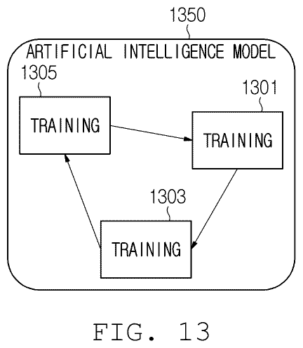

FIG. 12 shows an example of an artificial intelligence model for a side effect grade system according to an embodiment of the present disclosure. Referring to FIG. 12, the artificial intelligence model 1250 for the side effect grade system takes the form of multiple inputs and a single output. Accordingly, when input data 1202 including BDSI, ISD, IIPD, IISD, and ADMET is input, output data 1204 indicating one side effect grade is output. The artificial intelligence model 1250 predicts the grade class by analyzing the patterns of independent variables such as BDSI, ISD, IIPD, IISD, and ADMET of drug pairs. In addition, as shown in FIG. 13, the artificial intelligence model 1250 may independently verify (1303) the prediction (1301) results and proceed with further training (1305) while feeding back the verification contents. For example, if the prediction result is [symbol] Drug_1, Drug_2→Class ‘1’ [symbol] , the interpretation is [symbol] Drug 1 and Drug 2 have a ‘moderate’ probability of side effects occurring when used together.

[symbol]

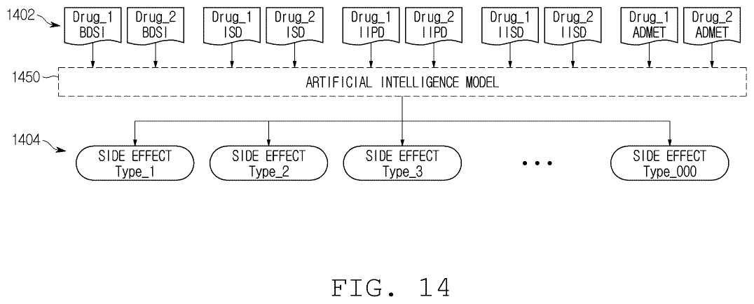

FIG. 14 shows an example of an artificial intelligence model for a side effect type system according to an embodiment of the present disclosure. Referring to FIG. 14, the artificial intelligence model 1450 for the side effect type system takes the form of multiple inputs and multiple outputs. Accordingly, when input data 1402 including BDSI, ISD, IIPD, IISD, and ADMET is input, output data 1404 indicating the type of side effect expressed by a plurality of items is output. The artificial intelligence model 1450 analyzes the patterns of independent variables such as BDSI, ISD, IIPD, IISD, and ADMET of drug pairs and predicts type classes. For example, if the prediction result is [symbol] Drug_1(subject_drug), Drug_2(affected_drug)→Type: ‘increase’, Type: ‘CNS depression’, Type: ‘hypotesion’ [symbol] , the interpretation is [symbol] Drug 1 has a probability that the side effects of ‘central nervous system depression’ and ‘low blood pressure’ ‘increase’ when used together with Drug 2 due to the effect of Drug 2.

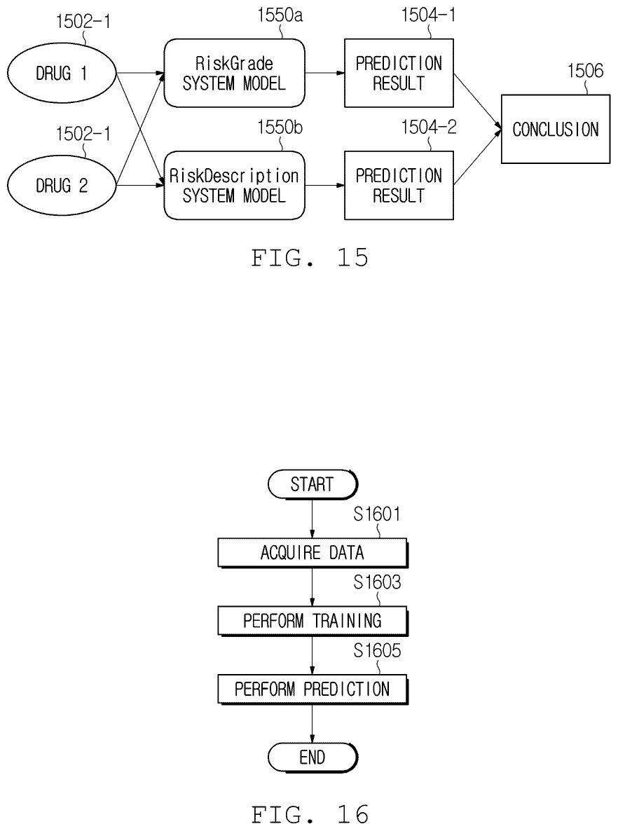

FIG. 15 shows a procedure for analyzing drug-drug interaction in a system according to an embodiment of the present disclosure. Referring to FIG. 15, a pair of drugs (e.g., Drug 1 1502-1, Drug 2 1502-2) for which the grade and type of side effects are to be predicted are input to a RiskGrade system model 1550a for determining the grade and a RiskDescription system model 1550b for determining the type. The input data includes attribute data used for training of the model, such as BDSI, ISD, IIPD, IIPDSP, and ADMET of the drug. The RiskGrade system model 1550a and the RiskDescription system model 1550b for determining the type output prediction results 1504-1 and 1504-2, respectively. Based on the prediction results 1504-1 and 1504-2, a conclusion 1506 predicting the grade and type of side effects between drugs is obtained.

The RiskDescription system model 1550b also predicts directionality, providing information on how the side effects of one drug change under the influence of another. For example, the RiskGrade system model 1550a may output a prediction result of [symbol] Drug_1, Drug_2→Class ‘1’ [symbol] , and the RiskDescription system model 1550b may output a prediction result of [symbol] Drug_1 (subject_drug), Drug_2 (affected_drug)→Type: ‘increase’, Prediction results of Type: ‘CNS depression’ and Type: ‘hypotesion’. In this case, the conclusion 1506 is [symbol] There is a ‘moderate’ probability of side effects occurring when drug 1 and drug 2 are used together, and the side effects of ‘central nervous system depression’ and ‘low blood pressure’ of drug 1 ‘increase’ due to the influence of drug 2.

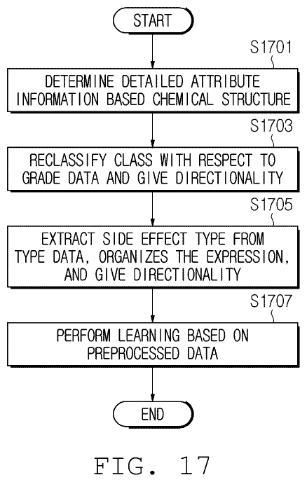

FIG. 16 shows an example of a procedure for training and prediction in a system according to an embodiment of the present disclosure. In the following description, the operating subject is described as a ‘device’, but the operations described later may be performed by a server or a user device.

Referring to FIG. 16, in step S1601, the device acquires data. The data is data for training and may include, for example, a drug chemical structure data set, a side effect grade data set between drugs, and a side effect type data set between drugs.

In step S1603, the device performs training. To perform training, the device may perform preprocessing on data and perform training using the preprocessed data.

In step S1605, the device performs prediction. That is, the device obtains output data including a prediction result from input data including information about a drug pair to be analyzed using a learned artificial intelligence model. At this time, the device may preprocess information about the drug pair into a format capable of being input to the artificial intelligence model.

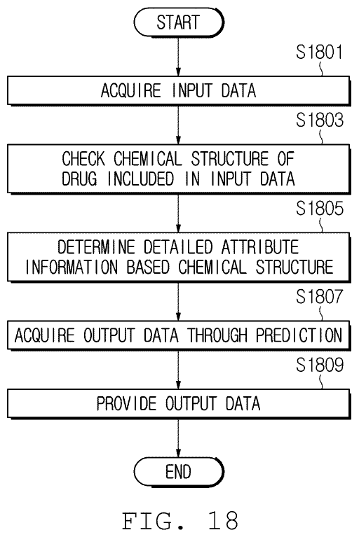

FIG. 17 shows an example of a procedure for performing training in a system according to an embodiment of the present disclosure. In the following description, the operating subject is described as a ‘device’, but the operations described later may be performed by a server or a user device.

Referring to FIG. 17, in step S1701, the device determines detailed attribute information based on the chemical structure. That is, as preprocessing for drug chemical structure data, the device generates detailed attribute information, for example, at least one attribute information of BDSI, ISD, IIPD, IISD, and ADMET, based on the chemical structure of the drug.

In step S1703, the device reclassifies the class with respect to the class data and gives directionality. That is, as preprocessing for the side effect type data between drugs, the device reclassifies the grade class values in a predefined format. That is, the device normalizes grade information according to drug combinations collected from different sources into a unified format according to a predefined format. In addition, the device gives directionality by adding an item that changes the order of drug combinations to the data set.

In step S1705, the device extracts the side effect type from the type data, organizes the expression, and gives directionality. In other words, as preprocessing for side effect grade data between drugs, the device extracts keywords expressing the type from data in sentence format. In addition, the device replaces expressions in a synonym or similar word relationship with representative expressions. In addition, the device gives directionality by adding an item that changes the order of drug combinations to the data set.

In step S1707, the device performs training based on the preprocessed data. To this end, the device generates a data set that maps the drug's attribute combination and side effect type/grade by matching preprocessed data sets. Then, the device performs training using the generated data set. That is, the device performs training using training data that has drug attribute combinations as independent variables and side effect types/grades as dependent variables. In other words, the device performs training on the artificial intelligence model by using the drug attribute combination information labeled by side effect type/grade as training data. In other words, the device performs prediction using training data and then updates the weights of the artificial intelligence model through a backpropagation operation.

FIG. 18 shows an example of a procedure for performing prediction in a system according to an embodiment of the present disclosure. In the following description, the operating subject is described as a ‘device’, but the operations described later may be performed by a server or a user device.

Referring to FIG. 18, in step S1801, the device acquires input data. The input data may be input through an input unit provided in the device (e.g., an input device such as a keyboard, a port connectable to an external storage medium, an interface that receives a signal through a communication network, etc.).

In step S1803, the device checks the chemical structure of the drug included in the input data. At this time, if the input data does not include chemical structure information (e.g., SMILES) but includes drug identification information (e.g., name, ID, etc.), the device may retrieve the chemical structure corresponding to the identification information from an internal or external database. To this end, the device may access a database, transmit a request containing identification information of the drug, and then receive chemical structure information as a response. In step S1805, the device determines detailed attribute information of the drug based on the chemical structure. The device generates detailed attribute information, for example, at least one attribute information of BDSI, ISD, IIPD, IISD, and ADMET, based on the chemical structure of the drug.

In step S1807, the device obtains output data through prediction. The device uses an artificial intelligence model to generate output data containing prediction results from detailed attribute information of a pair of drugs. Here, the output data may include at least one of side effect grade data and side effect type data as the prediction result. According to another embodiment, the output data may be converted into text results capable of being more easily understood by users.

In step S1809, the device provides the output data. Here, the output data is provided to the user who requested or ordered analysis. For example, the output data may be visually output through an output unit (e.g., screen, etc.) provided in the device, or may be transmitted to an external device.

While the exemplary methods of the present disclosure described above are represented as a series of operations for clarity of description, it is not intended to limit the order in which the steps are performed, and the steps may be performed simultaneously or in different order as necessary. In order to implement the method according to the present disclosure, the described steps may further include other steps, may include remaining steps except for some of the steps, or may include other additional steps except for some of the steps.

The various embodiments of the present disclosure are not a list of all possible combinations and are intended to describe representative aspects of the present disclosure, and the matters described in the various embodiments may be applied independently or in combination of two or more.

In addition, various embodiments of the present disclosure may be implemented in hardware, firmware, software, or a combination thereof. In the case of implementing the present invention by hardware, the present disclosure can be implemented with application specific integrated circuits (ASICs), Digital signal processors (DSPs), digital signal processing devices (DSPDs), programmable logic devices (PLDs), field programmable gate arrays (FPGAs), general processors, controllers, microcontrollers, microprocessors, etc.

The scope of the disclosure includes software or machine-executable commands (e.g., an operating system, an application, firmware, a program, etc.) for enabling operations according to the methods of various embodiments to be executed on an apparatus or a computer, a non-transitory computer-readable medium having such software or commands stored thereon and executable on the apparatus or the computer.

1. A method of analyzing drug-drug interaction (DDI), the method comprising:
acquiring a first data set for chemical structures of drugs, a second data set for a grade of a side effect between the drugs and a third data set for a type of a side effect between the drugs, as data sets for training, at a processor configured to analyze the DDI;
generating detailed attribute information of each of the drugs, by preprocessing the first data set, at the processor;
standardizing a class included in the second data set and giving directionality, by preprocessing the second data set, at the processor;
extracting expressions representing a side effect type included in the third data set, normalizing the expressions and giving directionality to the third data set, by preprocessing the third data set, at the processor;
training at least one artificial intelligence model stored in a memory using the preprocessed first data set, the preprocessed second data set and the preprocessed third data set, at the processor; and
determining the grade and type of the side effect of a pair of drugs from information on the pair of drugs using the at least one artificial intelligence model, at the processor.

2. The method of claim 1, wherein the training the at least one artificial intelligence model comprises generating a training data set mapping the grade and type of the side effect to an attribute combination of the drug, by matching the preprocessed first set data with the preprocessed second data set and the preprocessed third data set.

3. The method of claim 1, wherein the determining the grade and type of the side effect between the pair of drugs from the information on the pair of drugs comprises:
generating detailed attribute information of each of the pair of drugs, by preprocessing the information on the pair of drugs, at the processor; and
inputting the detailed attribute information as input data of the at least one artificial intelligence model, at the processor.

4. The method of claim 1,
wherein the detailed attribute information comprises BDSI (Binary data of Drug Structural Information), ISD (Index of Similarity between Drugs), IIPD (Index of Interaction between Protein and Drug), IISD (Index of Interaction Similarity between Drugs), and ADMET (Absorption Distribution Metabolism Excretion Toxicity) of each drug.

5. The method of claim 1,
wherein the second data set comprises side effect grade data between first drugs collected from a first source and side effect grade data between second drugs from a second source,
wherein the side effect grade data between the first drugs and the side effect grade data between the second drugs indicate the same class with different expressions, and
wherein the different expressions indicating the same class are normalized through the preprocessing.

6. The method of claim 1,
wherein the third data set comprises a first sentence expressing a type of a side effect of a first pair of drugs and a second sentence expressing a type of a side effect of a second pair of drugs,
wherein each of the first sentence and the second sentence comprises an expression indicating at least one type,
wherein the first sentence and the second sentence comprise different expressions indicating the type of the same meaning, and
wherein the different expressions indicating the type of the same meaning are replaced with a single term through the preprocessing.

7. The method of claim 1,
wherein the second data set comprises an item including side effect grade information of a pair of drugs combined in order of a first drug and a second drug, and
wherein the preprocessed second data set is processed to further include side effect grade information of the pair of drugs combined in order of the second drug and the first drug by giving the directionality.

8. The method of claim 1,
wherein the third data set comprises an item including side effect type information of a pair of drugs combined in order of a first drug and a second drug, and
wherein the preprocessed third data set is processed to further include side effect type information of the pair of drugs combined in order of the second drug and the first drug by giving the directionality.

9. The method of claim 1, wherein the at least one artificial intelligence model comprises a first artificial intelligence model of multiple input and a single output predicting the side effect grade and a second artificial intelligence model of multiple input and multiple output predicting the side effect type.

10. The method of claim 1, further comprising transmitting data indicating a grade and type of a side effect between the pair of drugs to another device.

11. A device for analyzing drug-drug interaction (DDI), the device comprising:
a memory configured to store at least one artificial intelligence model; and
a processor connected to the memory,
wherein the processor is configured to:
acquire a first data set for chemical structures of drugs, a second data set for a grade of a side effect between the drugs and a third data set for a type of a side effect between the drugs, as data sets for training;
generate detailed attribute information of each of the drugs, by preprocessing the first data set;
normalizing a class included in the second data set and giving directionality, by preprocessing the second data set;
extract expressions representing a side effect type included in the third data set, normalizing the expressions and giving directionality to the third data set, by preprocessing the third data set;
learn the at least one artificial intelligence model using the preprocessed first data set, the preprocessed second data set and the preprocessed third data set; and
determine the grade and type of the side effect of a pair of drugs from information on the pair of drugs using the at least one artificial intelligence model.
